SEQUENCE LISTING (31 sequences)

SEQ ID NO: 1 - DNA, 563 bp, Artificial sequence
  Synthesized using sequential PCR techniques
ggatatccaccatggacttcgggttgagcttggttttccttgtccttactttaaaaggtgtccagtgtgatagccacgctaagcggcaccacggatataagcggaagttccacgagaagcaccactcgcacagaggatactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 2 - protein, 24 aa, Artificial sequence
  Synthesized using sequential PCR techniques
DSHAKRHHGYKRKFHEKHHSHRGY

SEQ ID NO: 3 - protein, 16 aa, Artificial sequence
  Synthesized using sequential PCR techniques
SGGGGSGGGGSGGGGS

SEQ ID NO: 4 - protein, 165 aa, Artificial sequence
  Synthesized using sequential PCR techniques
DSHAKRHHGYKRKFHEKHHSHRGYSGGGGSGGGGSGGGGSDVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 5 - DNA, 533 bp, Artificial sequence
  Synthesized using squential PCR techniques
ggatatccaccatggacttcgggttgagcttggttttccttgtccttactttaaaaggtgtccagtgtaagcggctgtttaaggagctcaagttcagcctgcgcaagtactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 6 - protein, 14 aa, Artificial sequence
  Synthesized using squential PCR techniques
KRLFKELKFSLRKY

SEQ ID NO: 7 - protein, 155 aa, Artificial sequence
  Synthesized using squential PCR techniques
KRLFKELKFSLRKYSGGGGSGGGGSGGGGSDVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 8 - DNA, 89 bp, Artificial sequence
  Primer 986
caccactcgcacagaggatactctggtggcggtggctcgggcggaggtgggtcgggtggcggcggatccgacgtgaagcttgtggagtc

SEQ ID NO: 9 - DNA, 84 bp, Artificial sequence
  Primer 987
ggtgtccagtgtgatagccacgctaagcggcaccacggatataagcggaagttccacgagaagcaccactcgcacagaggatac

SEQ ID NO: 10 - DNA, 74 bp, Artificial sequence
  Primer 988
gatatccaccatggacttcgggttgagcttggttttccttgtccttactttaaaaggtgtccagtgtgatagcc

SEQ ID NO: 11 - DNA, 87 bp, Artificial sequence
  Primer 989
gttcagcctgcgcaagtactctggtggcggtggctcgggcggaggtgggtcgggtggcggcggatccgacgtgaagcttgtggagtc

SEQ ID NO: 12 - DNA, 69 bp, Artificial sequence
  Primer 990
gtccttactttaaaaggtgtccagtgtaagcggctgtttaaggagctcaagttcagcctgcgcaagtac

SEQ ID NO: 13 - DNA, 65 bp, Artificial sequence
  Primer 991
ggatatccaccatggacttcgggttgagcttggttttccttgtccttactttaaaaggtgtccag

SEQ ID NO: 14 - DNA, 39 bp, Artificial sequence
  Primer 452
tgggtcgacwgatggggstgttgtgctagctgaggagac

SEQ ID NO: 15 - protein, 18 aa, Artificial sequence
  Protegrin PG-1
RGGRLCYCRRRFCVCVGR

SEQ ID NO: 16 - DNA, 57 bp, Artificial sequence
  Protegrin PG-1
aggggaggtcgcctgtgctattgtaggcgtaggttctgcgtctgtgtcggacgagga

SEQ ID NO: 17 - protein, 18 aa, Artificial sequence
  Novispirin G10
KNLRRIIRKGIHIIKKYG

SEQ ID NO: 18 - DNA, 36 bp, Artificial sequence
  Forward primer 1
ggtggttgctcttccaacaggggaggtcgcctgtgc

SEQ ID NO: 19 - DNA, 23 bp, Artificial sequence
  Reverse primer 2
ccggatcctcgtccgacacagac

SEQ ID NO: 20 - DNA, 23 bp, Artificial sequence
  Forward primer 3
ggggatccggtggcggtggctcg

SEQ ID NO: 21 - DNA, 26 bp, Artificial sequence
  Reverse primer 4
aacatcgatagatccgccgccacccg

SEQ ID NO: 22 - DNA, 23 bp, Artificial sequence
  Forward primer 5
ggatcgatgttgtgatgacccag

SEQ ID NO: 23 - DNA, 31 bp, Artificial sequence
  Reverse primer 6
gcgggtcgaccgacttacgtttcagctccag

SEQ ID NO: 24 - DNA, 29 bp, Artificial sequence
  Forward primer 7
gcgggtcgacgtgaagctggtggagtctg

SEQ ID NO: 25 - DNA, 30 bp, Artificial sequence
  Reverse primer 8
gggtgttgagctagctgaagagacggtgac

SEQ ID NO: 26 - protein, 24 aa, Artificial sequence
  Linker 2
LDPKSCERSHSCPPCGGGSGGGTS

SEQ ID NO: 27 - DNA, 72 bp, Artificial sequence
  Linker 2
ctcgacccaaagagctgcgagcggagccacagctgcccaccgtgcgggggtgggtccggcggtggcactagt

SEQ ID NO: 28 - DNA, 28 bp, Artificial sequence
  Forward primer 9
gtgggctagcctcgacccaaagagctgc

SEQ ID NO: 29 - DNA, 38 bp, Artificial sequence
  Reverse primer 10
aggttctcggggctgcccactagtgccaccgccggacc

SEQ ID NO: 30 - DNA, 19 bp, Artificial sequence
  Forward primer 11
gggcagccccgagaacaac

SEQ ID NO: 31 - DNA, 33 bp, Artificial sequence
  Reverse primer 12
ggtggtctgcagtttacccggggacagggagag

(19) United States
(12) Patent Application Publication
(10) Pub. No.: US 2003/0143234 A1
(43) Pub. Date: Jul. 31, 2003

(54) Anti-microbial targeting chimeric pharmaceutical

(76) Inventors: Wenyuan Shi, Los Angeles, CA (US); Sherie L. Morrison, Los Angeles, CA (US); Kham Trinh, Alhambra, CA (US); Letitia Wims, Culver City, CA (US); Li Chen, Los Angeles, CA (US); Maxwell Anderson, Seattle, WA (US); Fengxia Qi, Harbor City, CA (US)

Correspondence Address: GRAY CARY WARE & FREIDENRICH LLP, 153 TOWNSEND, SUITE 800, SAN FRANCISCO, CA, 94107, US

(21) Appl. No.: 10/077,624

(22) Filed: Feb. 14, 2002

Related U.S. Application Data
(63) Continuation-in-part of application No. 09/910,358, filed on Jul. 19, 2001.
Application No. 09/910,358 is a continuation-in-part of application No. 09/378,577, filed on Aug. 20, 1999.

(51) Int. Cl. A61K 39/40; C07K 16/46

(52) U.S. Cl. 424/178.1; 530/391.1

(57) ABSTRACT

The present invention is based on the discovery of a composition that provides targeted anti-microbial effect. Specifically the composition contains a targeting moiety which recognizes a target microbial organism and an anti-microbial peptide moiety which has anti-microbial activity. In addition, the present invention provides methods of treating a microbial infection, e.g., on mucosal surfaces by using the compositions provided by the present invention.

### CROSS REFERENCE TO RELATED APPLICATIONS

This application is a continuation-in-part of U.S. application Ser. No. 09/910,358, which is a continuation-in-part of U.S. application Ser. No. 09/378,577, all of which are incorporated herein by reference.

### FIELD OF THE INVENTION

This invention relates generally to the field of anti-microbial treatment, and more specifically to targeted anti-microbial treatment by chimeric constructs.

### BACKGROUND OF THE INVENTION

The Centers for Disease Control estimates that half of more than 100 million annual prescriptions of antibiotics are unnecessary. As a result, microbes have, in many cases, adapted and are resistant to antibiotics due to constant exposure and improper use of the drugs. It is estimated that the annual cost of treating drug resistant infections in the United States is approximately $5 billion. This continued emergence of anti-microbial-resistant bacteria, fungi, yeast and parasites has encouraged efforts to develop other agents capable of killing pathogenic microbes. Furthermore, there are urgent needs for target-specific anti-microbial agents since many microbial pathogens reside with non-harmful commensal bacteria that are important for optimum health.

Recent researches have revealed a class of naturally occurring anti-microbial peptides in humans, other mammals, insects and other organisms. A negative aspect of treatment with antibiotics or anti-microbial peptides is their ability to kill or inhibit the growth of a broad spectrum of organisms. The human body is home to tens of thousands of different bacteria, many of which are vital for optimum health. Overuse of antibiotics can seriously disrupt the normal ecology of the body and render humans more susceptible to bacterial, yeast, viral, and parasitic infection. This effect is also seen with administration of anti-microbial peptides. For example, histatin has been shown to kill not only gram-positive bacteria responsible for dental caries, but also non-harmful commensal gram-positive bacteria in oral cavity, thus general administration of histatin can actually cause undesirable effect by stimulating the growth of gram-negative bacteria, such as Actinobacillus sp or Fusobacterium sp, many of which may cause periodontal diseases. Accordingly, histatin is not useful by itself for prevention of dental disease.

Another disadvantage of administration of anti-microbial peptides is their ability to damage host cells at higher concentrations since these positively charged peptides can also penetrate and disrupt eukaryotic cell membranes.

Previous efforts to target delivery of pharmaceutically active agents relied principally on non-specific chemical reactions between a pharmaceutically active agent, and a targeting component. For example Shih et al. U.S. Pat. No. 5,057,313 refers to targeting delivery of drugs, toxins and chelators to specific sites in an organism by loading a therapeutic or diagnostic component onto a polymeric carrier, followed by conjugation of the carrier to a targeting antibody. Hansen, U.S. Pat. No. 5,851,527 claims a similar invention.

A drawback to this approach is that the non-specific linkage of the pharmaceutical reagents to unknown sites on the antibody molecule used for targeting may interfere with delivery of the therapeutic agents. See Rodwell et al., U.S. Pat. No. 4,671,958. Moreover, chemical modification of a targeting antibody by the nonspecific reactions during conjugation may substantively alter the antibody itself, thereby affecting its binding to targets. Chemical linkage is very inefficient, and the result is non-uniform, making the technique very difficult to use in practice.

More recently, there have been a number of reports of the use of recombinant techniques to produce fusion proteins for the treatment of disease. See Penichet and Morrison, J. Immunological Methods, 248:91-101 (2001) for review. Penichet et al. discuss efforts to treat malignant disease using a genetically engineered protein construct including an immunological component that binds specifically to tumor cells and a cytokine capable of eliciting significant antitumor activity. See, e.g. Pastan et al U.S. Pat. No. 5,981,726, and Fell, Jr. et al., U.S. Pat. No. 5,645,835.

However, to date there have not been any reports of directing anti-microbial agents to affected regions of humans or animals using target-specific molecules. There is a need in the art to provide methods and compositions useful for treatment of microbial organisms and microbially mediated diseases, especially microbial diseases of mucosal surfaces that are not readily accessible by normal anti-microbial mechanisms provided by the immune systems.

### SUMMARY OF THE INVENTION

The present invention is based on the discovery that anti-microbial peptides can be specifically targeted to desired target microbial organisms by a targeting moiety connected to the anti-microbial peptides. Accordingly the present invention provides a composition that has an anti-microbial effect on a target microbial organism. The present invention also provides methods of treating a microbial infection, e.g., on mucosal surfaces by using the compositions provided by the present invention.

In one embodiment, the present invention provides a composition useful for treatment of microbial organisms. The composition comprises a targeting moiety and an anti-microbial peptide moiety, wherein the targeting moiety is coupled to the anti-microbial peptide moiety and recognizes a target microbial organism and wherein the composition has an anti-microbial effect on the target microbial organism.

In another embodiment, the composition comprises a targeting moiety and an anti-microbial peptide moiety, wherein the targeting moiety is a peptide, e.g., polypeptide or small peptide and is fused in-frame with the anti-microbial peptide moiety. Such composition can be produced recombinantly using an expression system, e.g., bacterial, yeast, or eukaryotic cell expression system, without having to deal with problems associated with chemical or physical linkages.

In another embodiment, the present invention provides a method of treating a target microbial organism infection. The method comprises administering to a subject in need of such treatment an effective amount of the composition of the present invention.

### SUMMARY OF THE FIGURES

FIG. 1 shows a schematic diagram of the sequential PCR reactions used to assemble the heavy chain portion of the antibody-based fusion protein.

FIG. 2 shows the sequences (SEQ ID NOS: 8-14) of the primers used in the sequential PCR reactions in embodiments of the present invention.

FIG. 3 shows the nucleotide sequence (SEQ ID NO: 1) encoding the anti-microbial peptide, histatin 5, the linker peptide, and the variable region of the heavy chain derived from the SWLA3 monoclonal antibody together with the amino acid sequence (SEQ ID NO: 4).

FIG. 4 shows the nucleotide sequence (SEQ ID NO: 5) encoding the anti-microbial peptide, dhvar 1, the linker peptide, and the variable region of the heavy chain derived from the SWLA3 monoclonal antibody together with the amino acid sequence (SEQ ID NO: 7).

FIG. 5 shows the schematic diagram of making a minibody-anti-microbial peptide fusion protein.

### DESCRIPTION OF THE PREFERRED EMBODIMENTS

The present invention relates in general to the targeted anti-microbial effects using a composition, e.g., a chimeric construct containing a targeting moiety and an anti-microbial peptide moiety. The present invention also provides methods of treating a microbial infection using the compositions provided by the present invention.

According to the present invention, a targeting moiety can be any suitable structure that recognizes and binds to a target microbial organism. For example, a targeting moiety can be a polypeptide, peptide, small molecule, ligand, receptor, antibody, protein, or portions thereof that specifically interacts with a target microbial organism, e.g., the cell surface appendages such as flagella and pili, and surface exposed proteins, lipids and polysaccharides of a target microbial organism.

In one embodiment, the targeting moiety of the present invention is a monoclonal antibody or various forms of a monoclonal antibody that specifically recognize an epitope or antigen of a target microbial organism. Such epitope or antigen usually is species-specific and located on the surface of a target microbial organism. A monoclonal antibody or various forms thereof in a targeting moiety can direct an anti-microbial peptide moiety to its target site. Furthermore, it may also provide anti-microbial effect in addition to the effect provided by the anti-microbial peptide moiety since such monoclonal antibody may engage an immune system and elicit an antibody-associated immune response, e.g., humoral immune response.

A monoclonal antibody specific to a microbial organism can be made using any methods readily available to one skilled in the art. For example, as described in the U.S. Pat. No. 6,231,857 (incorporated herein by reference) three monoclonal antibodies, i.e., SWLA1, SWLA2, and SWLA3 have been made against S. mutans. Monoclonal antibodies obtained from non-human animals to be used in a targeting moiety can also be humanized by any means available in the art to decrease their immunogenicity and possibly increase their ability to elicit anti-microbial immune response of a human.

Various forms of a monoclonal antibody include, without limitation, scFv, minibody, Di-miniantibody, Tetra-miniantibody, (scFv)₂, Diabody, scDiabody, Triabody, Tetrabody, and Tandem diabody. A scFv usually comprises a single chain containing the variable regions of a light chain and a heavy chain. A minibody usually comprises the variable regions of a light chain and a heavy chain, e.g., scFv joined to a heavy chain constant region, e.g., about 20 amino acids or the third constant domain, C_{H}3 domain, either directly or via a linker, e.g., about 10 to 25 amino acids. A minibody can be readily made by expressing its encoding sequence in any suitable cell lines, e.g. Sp2/0 cells. A readily prepared version of a minibody usually forms a disulfide-linked dimer by virtue of the constant region, e.g., C_{H}3 domain and a cysteine-containing linker. Various forms of a monoclonal antibodies are described in Little et al., Immunology Today, 21:364-370 (2000), which is incorporated herein by reference.

Alternatively, the targeting moiety of the present invention can include all or a portion of one or more variable regions that are capable of specifically recognizing or binding to a target microbial organism and optionally a portion of constant regions that is sufficient for dimerization. For example, the variable region of a heavy chain has three complementarity determining regions (CDRs) and are capable of binding to an antigen. One skilled in the art can readily assess the minimum variable regions required of any particular monoclonal antibody for antigen or epitope binding.

According to another embodiment of the present invention, a targeting moiety can be a peptide identified through screening peptide or small molecule libraries. For example, a phage display peptide library can be screened against a target microbial organism or a desired antigen or epitope thereof. Any peptides identified through such screening can be used as a targeting moiety for the target microbial organism.

The targeting moiety of the present invention can also be a ligand, receptor, or fragment thereof that specifically recognizes a target microbial organism. For example, glucan binding proteins of Streptococcus mutans that can specifically bind insoluable glucans on the surface of S. mutans.

The composition of the present invention can contain one or more targeting moieties capable of targeting the same or different target microbial organisms. In one embodiment, the composition of the present invention contains one or more targeting moieties capable of targeting different sites or structures of the same target microbial organism. Such composition is useful for preventing resistance of a target microbial organism to the composition.

According to the present invention, an anti-microbial peptide moiety of the composition of the present invention comprises one or more anti-microbial peptides. In general, any known or later discovered anti-microbial peptides can be used for the compositions of the present invention. Anti-microbial peptides are various classes of peptides, e.g., peptides originally isolated from plants as well as animals. In animals, anti-microbial peptides are usually expressed by various cells including neutrophils and epithelial cells. In mammals including human, anti-microbial peptides are usually found on the surface of the tongue, trachea, and upper intestine.

Naturally occurring anti-microbial peptides are generally amphipathic molecules that contain fewer than 100 amino acids. Many of these peptides generally have a net positive charge (i.e., cationic) and most form helical structures. It is generally believed that these peptides' anti-microbial efficacy is in their ability to penetrate and disrupt the microbial membranes, thereby killing the microbe or inhibiting its growth.

The anti-microbial activities of the anti-microbial peptides of the present invention include, without limitation, antibacterial, antiviral, or antifungal activities. For example, one well-known class of anti-microbial peptides is the tachyplesins which are described as having antifungal and antibacterial activities. Andropin, apidaecin, bactencin, clavanin, dodecappeptide, defensin, and indolicidin are anti-microbial peptides having antibacterial activities. Buforin, nisin and cecropin peptides have been demonstrated to have anti-microbial effects on Escherichia. coli,, Shigella disenteriae, Salmonella typhimurium, Streptococcus pneumoniae, Staphylococcus aureus, and Pseudomonas aeroginosa. Magainin and ranalexin peptides have been demonstrated to have anti-microbial effects on the same organsims, and in addition have such effects on Candida albicans, Cryptococcus neoformans, Candida krusei, and Helicobacter pylori. Magainin has also been demonstrated to have anti-microbial effects on herpes simplex virus. Alexomycin peptides have been demonstrated to have anti-microbial effects on Camphylobacter jejuni, Moraxella catarrhalis and Haemophilus inflluenzae while α defensin and β pleated sheet defensin peptides have been shown to have anti-microbial effects on Streptococcus pneumoneae.

Histatin peptides and the derivatives thereof are another class of anti-microbial peptides, which have antifungal and antibacterial activities against a variety of organisms including Streptococcus mutans. MacKay, B. J. et al., Infect. Immun. 44:695-701 (1984); Xu, et al., J. Dent. Res. 69:239 (1990).

In one embodiment, the anti-microbial peptide moiety of the present invention contains one or more anti-microbial peptides from a class of histatin peptides and the derivatives thereof. For example, the anti-microbial peptide moiety of the present invention contains one or more derivatives of histatin including, without limitation, histatin 5 having an amino acid sequence as shown in SEQ ID NO. 2 or dhvar 1 having an amino acid sequence as shown in SEQ ID NO. 6.

In another embodiment, the anti-microbial peptide moiety of the present invention contains one or more anti-microbial peptides from a class of protegrins and the derivatives thereof. For example, the anti-microbial peptide moiety of the present invention contains protegrin PG-1 having an amino acid sequence RGGRLCYCRRRFCVCVGR as shown in SEQ ID NO. 15. The protegrin peptides have been shown to have anti-microbal effects on Streptococcus mutans, Neisseria gonorrhoeae, Chlamydia trachomatis and Haempohilus influenzae. Protegrin peptides are described in the U.S. Pat. Nos. 5,693,486, 5,708,145, 5,804,558, 5,994,306, and 6,159,936, all of which are incorporated herein by reference.

In yet another embodiment, the anti-microbial peptide moiety of the present invention contains one or more anti-microbial peptides from a class of novispirin and the derivatives thereof as described in Sawai et al., “Impact of Single—Residue Mutations on the Structure and Function of Ovispirin/Novispirin Antimicrobial Peptides.” Protein Engineering (in press). For example, the anti-microbial peptide moiety of the present invention contains novispirin G10 having an amino acid sequence KNLRRIIRKGIHIIKKYG as shown in SEQ ID NO. 17 for treating cariogenic organisms, e.g., Streptococcus mutans.

In still another embodiment, the anti-microbial peptide moiety contains one or more anti-microbial peptides including, without limitation, alexomycin, andropin, apidaecin, bacteriocin, β-pleated sheet bacteriocin, bactenecin, buforin, cathelicidin, α-helical clavanin, cecropin, dodecapeptide, defensin, β-defensin, α-defensin, gaegurin, histatin, indolicidin, magainin, nisin, protegrin, ranalexin, tachyplesin, and derivatives thereof.

The anti-microbial peptide moiety of the present invention can include one or more anti-microbial peptides, which can be the same or different anti-microbial peptides. The anti-microbial peptides of the present invention can also be modified, e.g., to enhance its anti-microbial effectiveness, its cell delivery, its compatibility with the rest of the composition structure, or the manipulation of the composition in production.

The targeting moiety and the anti-microbial peptide moiety of the present invention can be coupled by various means known to one skilled in the art. For example, the targeting moiety and the anti-microbial peptide moiety can be covalently coupled or connected by a peptide linker and the composition so formed can be constructed through molecular cloning and overexpressed or purified as one polypeptide unit in a bacterial, yeast, or eukaryotic cell expression system. Any peptide linker can be used to connect the targeting moiety and the anti-microbial peptide moiety of the present invention. In one embodiment, the peptide linker does not interfere or inhibiting the activity of the targeting moiety or the anti-microbial peptide moiety. In another embodiment, the peptide linker is from about 10 to 60 amino acids, from about 15 to 25 amino acids, or about 15 amino acids.

An anti-microbial peptide can be connected to a targeting moiety at either or both ends of the targeting moiety. In one embodiment, a targeting moiety is a peptide or polypeptide which can be fused in frame at N-terminal, C-terminal, or both ends with one or more anti-microbial peptides.

The composition of the present invention can be made by any suitable means known to one skilled in the art. For example, a nucleotide sequence encoding a targeting moiety ligated to a nucleotide sequence encoding an anti-microbial peptide moiety, either directly or via a nucleotide sequence encoding a peptide linker, can be expressed in an appropriate expression system, e.g., a commercially available bacterial, yeast, or eukaryotic cell expression system. Usually for expressing in a bacterial expression system, an autocatalytic protein, e.g., intein and a chitin-binding domain (CBD) are used for purification purpose. For expressing in a yeast expression system, a pheromon factor α is usually fused to the N-terminal of a coding sequence while a myocin-his tag is fused to the C-terminal of the coding sequence for easy handling of the expressed product during the purification process.

In one embodiment of the present invention, a commercially available yeast expression system is modified, e.g., proteins used for bacterial expression systems are used for yeast expression. For example, a sequence encoding the composition of the present invention is fused with a sequence encoding pheromon factor α and a sequence encoding intein and CBD and is expressed in a yeast expression system.

The compositions of the present invention can be used to treat any target microbial organisms. For example, the target microbial organism of the present invention can be any bacteria, rickettsia, fungi, yeasts, protozoa, or parasites. In one embodiment, the target microbial organism is a cariogenic organism, e.g., Streptococcus mutans.

In another embodiment, the target microbial organisms of the present invention include, without limitation, Escherichia. coli, Camphylobacter jejuni, Candida albicans, Candida krusei, Chlamydia trachomatis, Clostridium difficile, Cryptococcus neoformans, Haempohilus influenzae, Helicobacter pylor, Moraxella catarrhalis, Neisseria gonorrhoeae, Pseudomonas aeroginosa, Salmonella typhimurium, Shigella disenteriae, Staphylococcus aureus, and Streptococcus pneumoniae.

According to another feature of the present invention, the compositions of the present invention provide anti-microbial effect to target microbial organisms and can be used to treat a target microbial organism infection. An anti-microbial effect includes inhibiting the growth or killing of the target microbial organisms, or interfering with any biological functions of the target microbial organisms.

In general, the compositions of the present invention can be used to treat a target microbial organism infection at any place in a host, e.g., at any tissue. In one embodiment, the compositions of the present invention are used to treat a target microbial organism infection on a mucosal surface. A mucosal surface usually harbors a broad spectrum of microbial organisms and prefers a treatment that is least disturbing to the balance of the entire microbial organism population, e.g., specific to pathogenic microbial organisms and has minimum effect on the non-pathogenic microbial population. For example, in human mouth there usually exist many different microbes including yeasts and bacteria. A lot of bacteria are non-harmful commensal bacteria that are essential for maintaining a healthy and normal microbial flora to prevent the invasion and establishment of other pathogenic microbial organisms, e.g., yeast infection. Administering the composition of the present invention targets specifically to cariogenic organisms, e.g. Streptococcus mutans and will have minimum effect on non-targeted microbial organisms, thus will not have an undesirable effect by non-targeted microbial organisms.

A lot of places in an animal or human body have mucosal surfaces and can be treated with the compositions of the present invention to provide targeted anti-microbial effect. For example, mouth, vagina, gastrointestinal (GI) tract, esophageal tract, and respiratory tract, all of which can have microbial organism infection on its mucosal surfaces.

In particular, S. mutans infection is commonly found in mouth and causes dental caries. Porphyromonas gingivalis, various Actinomyces species, Veillonella, spirochetes, and gram-negative flora including black-pigmented bacteroides are commonly associated with infections of gingival and surrounding connective tissues, which cause periodontal diseases. Streptococcus pneumoniae, nontypeable Haemophilius influenza, or Moraxella cararrhalis infection is commonly found in acute otitis media (AOM) and otitis media effusion (OME) as complications of upper respiratory infections in young children.

Helicobacter pylori (H. pylori) bacteria are found in the gastric mucous layer or adherent to the epithelial lining of the stomach, and cause more than 90% of duodenal ulcers and up to 80% of gastric ulcers. Other GI tract infections include, without limitation, campylobacter bacterial infection, primarily Campylobacter jejuni associated with diarrhea, cholera caused by Vibrio cholerae serogroups, salmonellosis caused by bacteria salmonella such as S. Typhimurium and S. Enteritidis, shigellosis caused by bacteria Shigella, e.g., Shigella dysenteriae and traveler's diarrhea caused by enterotoxigenic Escherichia coli (ETEC). Clostridium difficile infection is also commonly found in gastrointestinal tract or esophageal tract.

Yeast or Candida infections (Candidiasis) typically occur either orally (Oropharyngeal Candida or OPC) or vaginally (Vulvovaginal Candida or VVC). Candidiasis is caused by a shift in the local environment that allows Candida strains (most commonly Candida albicans) already present on skin and on mucosal surfaces such as mouth and vagina to multiply unchecked. Gonorrhea, chlamydia, syphilis, and trichomoniasis are infections in the reproductive tract, which cause sexually transmitted diseases, e.g., pelvic inflammatory disease.

The compositions of the present invention can be administered to various mucosal surfaces, e.g., the mucosal surfaces described above, with each composition containing a targeting moiety corresponding to one or more specific microbial organisms of the infection, e.g., the microbial organisms described above.

The compositions of the present invention useful for treating target microbial organism infection can be administered alone, in a composition with a suitable pharmaceutical carrier, or in combination with other therapeutic agents. An effective amount of the compositions to be administered can be determined on a case-by-case basis. Usually the dosage required is lower than the dosage required for an anti-microbial peptide administered without being linked to a targeting moiety, e.g., 10⁻¹ lower. Factors to be considered usually include age, body weight, stage of the condition, other disease conditions, duration of the treatment, and the response to the initial treatment.

Typically, the compositions are prepared as a topical or an injectable, either as a liquid solution or suspension. However, solid forms suitable for solution in, or suspension in, liquid vehicles prior to injection can also be prepared. The composition can also be formulated into an enteric-coated tablet or gel capsule according to known methods in the art.

The compositions of the present invention may be administered in any way which is medically acceptable which may depend on the disease condition or injury being treated. Possible administration routes include injections, by parenteral routes such as intravascular, intravenous, intraepidural or others, as well as oral, nasal, ophthalmic, rectal, topical, or pulmonary, e.g., by inhalation. The compositions may also be directly applied to tissue surfaces. Sustained release, pH dependent release, or other specific chemical or environmental condition mediated release administration is also specifically included in the invention, by such means as depot injections or erodible implants.

In one embodiment, the compositions of the present invention are used to treat or prevent cariogenic organism infections, e.g., S. mutans infection associated with dental caries and are prepared as additives to food or any products having direct contact to an oral environment, especially an oral environment susceptible to dental caries. For example, to treat or prevent dental caries one or more compositions of the present invention can be formulated into a baby formula, mouthwash, lozenges, gel, varnish, toothpaste, toothpicks, tooth brushes, or other tooth cleansing devices, localized delivery devices such as sustained release polymers or microcapsules, oral irrigation solutions of any kind whether mechanically delivered or as oral rinses, pacifiers, and any food including, without limitation, chewing gums, candies, drinks, breads, cookies, and milk.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

### Example 1

### Construction and Expression of a Histatin 5 and Dhvar 1/SWLA3 Chimeric Antibody Fusion Protein With Activity Against S. mutans.

a. Construction of an Expression Vector for an Antibody-Based Fusion Protein

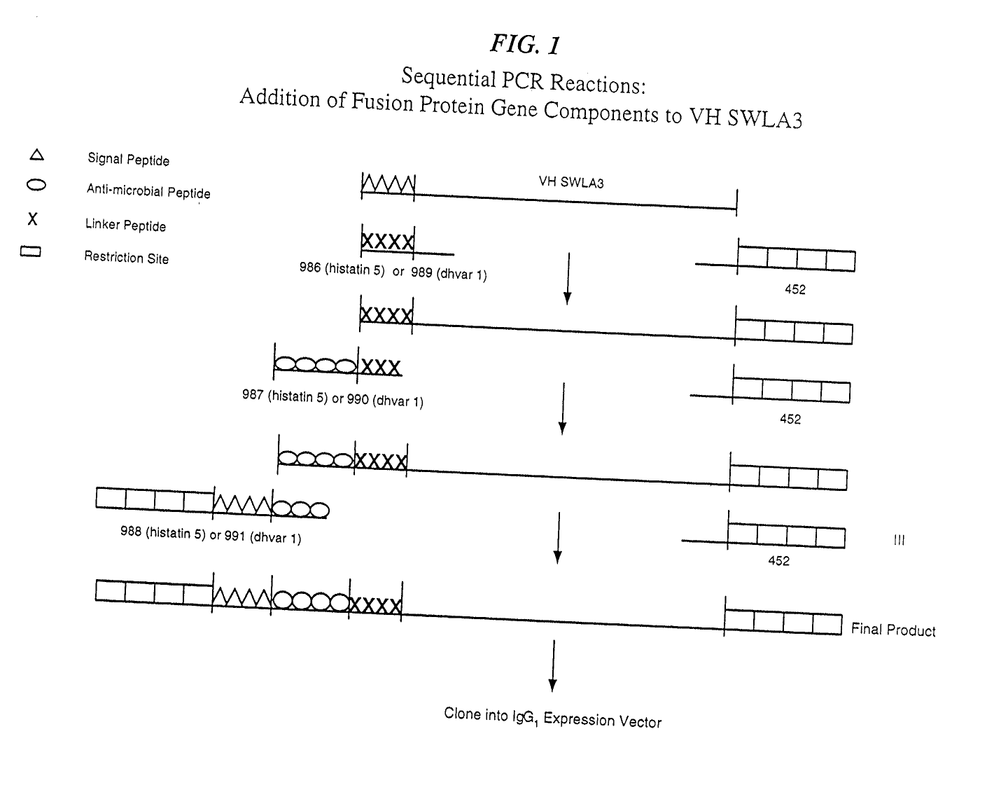

The construct that is ultimately cloned into an IgG₁ expression vector and leads to the expression of the targeted anti-microbial fusion protein was assembled according to the following method (see FIG. 1). The construct was assembled using sequential PCR and restriction enzymes techniques. The recognition sequence of the of the fusion protein was derived from heavy chain sequences of SWLA3, produced by hybridoma ATCC HB 12558. See Shi, U.S. Pat. No. 6,231,857, the disclosure of which is incorporated herein by reference, and U.S. patent application Ser. Nos. 09/378,577 and 09/881,823. Sequences encoding for histatin 5 or dhvar 1 were inserted upstream of the variable region of the heavy chain of SWLA3. The amino acid sequences used for histatin 5 and dhvar 1 are listed below:

Histatin 5 (SEQ ID NO: 2) DSHAKRHHGY KRKFHEKHHS HRGY

Dhvar 1 (SEQ ID NO: 6) KRLFKELKFS LRKY.

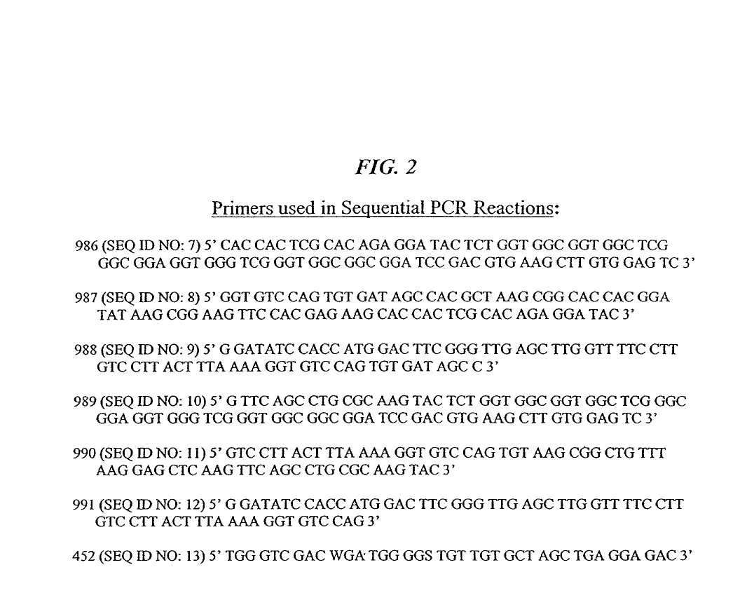

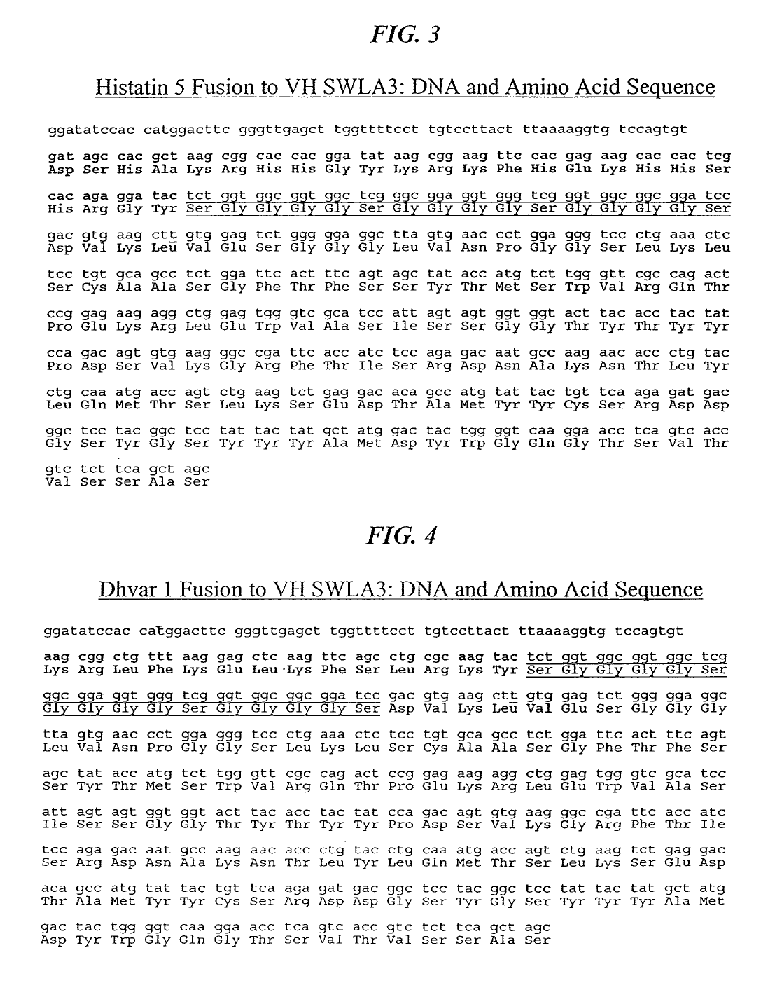

The source signal peptide was added upstream of the histatin 5 or dhvar 1, and a glycine/serine linker (SEQ ID NO: 3) was added to separate the fusion protein from the variable region of the heavy chain (VH) of the antibody. See FIG. 3 for the nucleic acid and encoded amino acid sequence for the histatin 5/SWLA3 V_{H} and FIG. 4 for the respective dhvar 1/SWLA3 V_{H} sequences. Sequential PCR reactions were used to complete the construct according to the following method (see FIG. 2 for the nucleic acid sequence of the primers used):

1. In the first PCR reaction a plasmid carrying the V_{H} of SWLA3 was used as the template with primer sets 986+452 (histatin 5) or 989+452 (dhvar 1). This reaction replaced the signal peptide in the original gene with the linker peptide at the 5′ end of the VH and inserted a restriction site at the 3′ end. The products of this reaction were isolated and used as a template in the second PCR reaction.

2. Using primer sets 987+452 (histatin 5) or 990+452 (dhvar 1) in the second PCR reaction added the anti-microbial peptide upstream from the linker peptide. The restriction site at the 3′ end was maintained. The products from this reaction were isolated and used as the template in the third PCR reaction.

3. With primer sets 988+452 (histatin 5) or 991+452 (dhvar 1) a signal peptide and restriction site were added upstream from the anti-microbial peptide. The restriction site at the 3′ end was maintained. Products from the third PCR were isolated.

4. Isolated products from the third PCR reaction were then cloned into Invitrogen's PCR2.1 vector via TOPO Cloning Kit and sequenced.

5. After the sequences of the two clones were confirmed, the inserts were moved into the IgG₁ PCR expression vector (pAH 4604) as an NheI/EcoRV fragment.

6. The final expression vectors for the histatin 5 and dhvar 1 antibody fusion proteins were named pAH 5993 and pAH 5994 respectively.

PCR conditions used were:

1. Denature @94° C. for 40 sec.

2. Anneal @60° C. for 40 sec.

3. Extend @72° C. for 40 sec.

4. Amplify for 30 cycles

5. Final Extension at 72° C. for 10 min.

FIG. 3 shows the nucleic acid sequence encoding the histatin 5 fusion to V_{H} SWLA3 and encoded amino acid sequence (SEQ ID NOS: 1 and 4) and FIG. 4 which shows the nucleic acid sequence encoding the dhvar 1 fusion to V_{H} SWLA3 and encoded amino acid sequence (SEQ ID NOS: 5 and 7). In the figures, the bold sequences represent the corresponding anti-microbial peptides, the underlined sequences represent the glycine/serine linker, and the single bolded underlined base in each sequence represents a silent point mutation. In the original sequence disclosed in Shi et al. U.S. patent application Ser. No. 09/881,823, the base is guanine.

The variable region of the light chain (V_{L}) from SWLA3 was cloned into a human kappa expression vector named 5940 pAG according to the method described in Shi et al. U.S. patent application Ser. No. 09/881,823. Briefly,

(i) DNA was prepared from the expression vectors and from the plasmid containing the correct V_{L}. See Current Protocols in Immunology, Section 2.12.1 (1994) for detailed information about the vectors that express the light and heavy chain constant regions.

(ii) The expression vector was digested with the appropriate restriction enzyme. The digests were then electrophoresed on an agarose gel to isolate the appropriate sized fragment.

(iii) The plasmid containing the cloned V_{L} region was also digested and the appropriate DNA fragment containing the V_{L} region was isolated from an agarose gel.

(iv) The V_{L} region and expression vector were then mixed together, T4 DNA ligase was added and the reaction mixture was incubated at 16° C. over night.

(v) Competent cells were transfected with the V_{L} ligation mixture and the clones expressing the correct ligation sequence were selected. Restriction mapping was used to confirm the correct structure.

b. Transfecting Eukaryotic Cells

Ten micrograms of DNA from each expression vector, pAH 5993 (histatin 5) or pAH 5994 (dhvar 1) and 5940 pAG, was linearized by BSPC1 (Stratagene, PvuI isoschizomer) digestion and 1×10⁷ myeloma cells (SP2/0 or P3×63.Ag8.653) were cotransfected by electroporation. Prior to transfection the cells were washed with cold PBS, then resuspended in 0.9 ml of the same cold buffer and placed in a 0.4 cm electrode gap electroporation cuvette. 960 microF and 200V was used for electroporation. The shocked cells were then incubated on ice in IMDM medium (Gibco, N.Y.) with 10% calf serum.

The transfected cells were plated into 96 well plates at a concentration of 10000 cells/well. Selective medium including selective drugs such as histidinol or mycophenolic acid were used to select the cells which contain expression vectors. After 12 days, the supernatants from growing clones were tested for antibody production.

c. Analyses of Histatin-5 and Dhvar 1 /SWLA3 Chimeric Antibody Fusion Proteins

ELISA assay was used to identify transfectomas that secrete the fusion IgG antibodies. 100 μl of 5 μg/ml goat anti-human IgG was added to each well of a 96-well ELISA plate and incubated overnight. The plate was washed several times with PBS and blocked with 3% BSA. Supernatants from above growing clones were added to the plate for 2 hours at room temperature to assay for their reactivity with goat anti-human Ig antibody. Plates were then washed and anti-human kappa antibody labeled with alkaline phosphatase diluted 1:10⁴ in 1% BSA was added for 1 hour at 37° C. Plates were washed with PBS and para-nitrophenyl phosphate in diethanolamine buffer (9.6% diethanolamine, 0.24 mM MgCl₂, pH 9.8) was added. Color development at OD₄₀₅ was indicative of cells producing H₂L₂.

For the supernatants that produce IgG constant regions, their reactivity with S. mutans was tested as described in Shi et al., Hybridoma 17:365-371 (1998). Briefly, bacteria strains listed in Table 1 were grown in various media suggested by the American Type Culture Collection. The anaerobic bacteria were grown in an atmosphere of 80% N₂, 10% CO₂, and 10% H₂ at 37° C. The specificity of antibodies to various oral bacteria was assayed with ELISA assays. Bacteria were diluted in PBS to OD₆₀₀=0.5, and added to duplicate wells (100 μl) in 96 well PVC ELISA plates preincubated for 4 h with 100 μl of 0.02 mg/ml Poly-L-lysine. These antigen-coated plates were incubated overnight at 4° C. in a moist box then washed 3 times with PBS and blocked with 0.5% fetal calf serum in PBS and stored at 4° C. 100 μl of chimeric antibodies at 50 μg/ml were added to the appropriate wells of the antigen plates, incubated for 1 h at RT, washed 3 times with PBS-0.05% Tween 20, and bound antibody detected by the addition of polyvalent goat-anti-human IgG antibody conjugated with alkaline phosphatase diluted 1:10³ with PBS-1% fetal calf serum. After the addition of the substrate, 1 mg/ml p-nitrophenyl phosphate in carbonate buffer (15 mM Na₂CO₃, 35 mM NaH₂CO₃, 10 mM MgCl₂ pH 9.6), the color development after 15 min was measured in a EIA reader at 405 nm. “+” means OD405>1.0; “−” means OD405<0.05. The negative control is <0.05. The results are given in Table 1.

TABLE 1
Reactivity of Antibody Fusion Proteins to Various Oral Bacterial Strains

| | | Hitstatin | Dhvar |
|---|---|---|---|
| | | 5/SWLA3 | 1/SWLA3 |
| | | Fusion | Fusion |
| Oral Bacteria | Strains | Antibodies | Antibodies |
| S. mutans | AATCC25175 | + | + |
| | LM7 | + | + |
| | OMZ175 | + | + |
| S. Mitis | ATCC49456 | − | − |
| S. rattus | ATCC19645 | − | − |
| S. sanguis | ATCC49295 | − | − |
| S. sobrinus | ATCC6715-B | − | − |
| S. sobrinus | ATCC33478 | − | − |
| L. acidophilus | ATCC4356 | − | − |
| L. casei | ATCC11578 | − | − |
| L. plantarum | ATCC14917 | − | − |
| L. salivarius | ATCC11742 | − | − |
| A. actinomycetemcomitans | ATCC33384 | − | − |
| A. naeslundi | ATCC12104 | − | − |
| A. viscosus | ATCC19246 | − | − |
| Fusobacterium nucleatum | ATCC25586 | − | − |
| Porphyromonas gingivalis | ATCC33277 | − | − |

The fusion proteins showed both specificity and anti-microbial efficacy against S. mutans. Like the monoclonal antibodies from which they are derived, the fusion proteins bind specifically to S. mutans. (See Table 1). They also have anti-bacterial efficacy against the bacteria, but are effective at a much lower concentration than histatin 5 alone. (See Table 2).

TABLE 2
Recombinant Histatin 5/SWLA3 fusion antibodies
targets S. mutans with a great sensitivity and specificity

Minimal Inhibitory Concentrations

| | S. mutans | S. sanguis | Host cells |
|---|---|---|---|
| Histatin 5 | ˜10 μM | ˜10 μm | >50 μM |
| Histatin 5/SWLA3 | ˜0.3 μM | ˜30 μM | >50 μM |
| fusion antibodies | | | |

This observation suggests that the recognition sequence is responsible for specific binding between the fusion protein and S. mutans, which locally enhances the concentration of histatin 5 at the bacterial cell surface. At the concentration at which the fusion protein showed antibacterial efficacy, the fusion proteins showed no inhibitory effect on other bacteria or host cells (Table 2). Accordingly, these results suggest that the basic design described herein may be useful for generating antibody-based fusion proteins for treatment of other infections and infestations.

### Example 2

### Construction And Analyses of A Chimeric Construct Containing Minibody And Anti-microbial Peptides

a. Construction of a Minibody-Peptide Fusion Protein

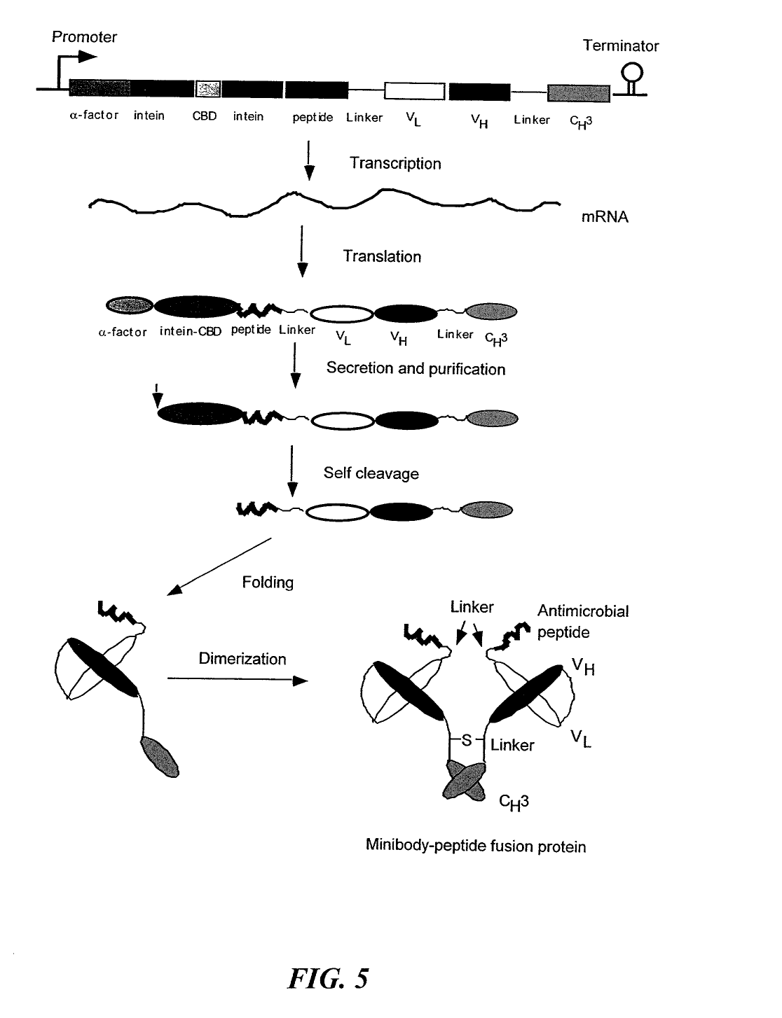

A minibody is a modified antibody molecular that comprises V_{L}-V_{H}-linker-Ch(1, 2, or 3) covalently linked in a head-to-tail fashion (see FIG. 5). To construct a minibody-anti-microbial peptide fusion protein, the anti-microbial peptide will be linked to the N-terminus of V_{L} via a poly glycine-serine linker peptide. The C-terminus of V_{L} will then be fused with the N-terminus of V_{H}, which then will be fused to a subdomain of the constant region (Ch) via another peptide linker. Inclusion of the subdomain from the constant region will ensure the efficient dimerization of the minibody in solution, and stabilize the effective conformation of the minibody. PCR and other DNA manipulation techniques will be used to piece together the DNA fragments encoding the anti-microbial peptide, the linkers, and the different domains of the minibody. Briefly, genes encoding the anti-microbial peptide, the linker, the different domains of the minibody will be synthesized by PCR using primers specific to the coding regions of the corresponding peptide or domain. Restriction enzyme cleavage site will be incorporated in the primers. After PCR, the DNA fragments will be digested with the appropriate restriction enzymes and ligated with T4 DNA ligase. Correct orientation of the DNA fragments will be ensured by incorporating different restriction sites at the different termini. The entire construct will be cloned into an appropriate expression vector and expressed in an appropriate host.

b. Construction of an Anti S. mutans Minibody-Protegrin Fusion Protein

The starting material for constructing the minibody will be the anti S. mutans monoclonal antibody, SWLA3, as described in the U.S. Pat. No. 6,231,857. The anti-microbial peptide will be protegrin as described in the U.S. Pat. Nos. 5,693,486, 5,708,145, 5,804,558, 5,994,306, and 6,159,936 and Zhao et al., FEBS lett, 1994, 346 (2-3): 285-8.

Synthesis of the protegrin gene fragment. The coding region of the protegrin will be synthesized as a DNA fragment with the following sequence: 5′-AGG GGA GGT CGC CTG TGC TAT TGT AGG CGT AGG TTC TGC GTC TGT GTC GGA CGA GGA-3′ (SEQ ID NO. 16). The fragment will be amplified by PCR using two primers:

Primer 1 (forward primer): 5′-GGT GGT TGC TCT TCC AAC AGG GGA GGT CGC CTG TGC-3′ (SEQ ID NO. 18); the underlined sequence is a Sap I restriction enzyme cleavage site.

Primer 2 (reverse primer): 5′-CCG GAT CCT CGT CCG ACA CAG AC-3′ (SEQ ID NO. 19); the underlined sequence is the Bam HI restriction site.

Amplification of the poly Ser-Gly linker region. The DNA encoding the poly-Ser-Gly linker will be amplified by PCR from the SWLA3-histatin construct using the following primers:

Primer 3 (forward): 5′-GG GGA TCC GGT GGC GGT GGC TCG-3′ (SEQ ID NO. 20); the underlined sequence is a Bam HI restriction site.

Primer 4 (reverse): 5′-AAC ATC GAT AGA TCC GCC GCC ACC CG-3′ (SEQ ID NO. 21); the underlined sequence is the Cla I restriction site.

Generation of the DNA fragment encoding the V_{L} region of SWLA3. The DNA fragment encoding the V_{L} region will be amplified by PCR using the following primers with the anti S. mutans monoclonal antibody SWLA3:

Primer 5 (forward): 5′-GG ATC GAT GTT GTG ATG ACC CAG-3′ (SEQ ID NO. 22); the underlined sequence is the Cla I restriction site.

Primer 6 (reverse): GCGG GTC GAC CGA CTT ACG TTT CAG CTC CAG-3′ (SEQ ID NO. 23); the underlined sequence is the Sal I restriction site.

Generation of the DNA fragment encoding the V_{H} region of SWLA3. The gene encoding the V_{H} region will be amplified by PCR using the following primers with the anti S. mutans monoclonal antibody SWLA3:

Primer 7 (forward): 5′-GCGG GTC GAC GTG AAG CTG GTG GAG TCT G-3′ (SEQ ID NO. 24); the underlined sequence is the Sal I restriction site.

Primer 8 (reverse): 5′-GGG TGT TGA GCT AGC TGA AGA GAC GGT GAC-3′ (SEQ ID NO. 25); the underlined sequence is the Nhe I restriction site.

Synthesis of the linker between V_{H} and C_{H}3. The amino acid sequence of the linker will be LDPKSCERSHSCPPCGGGSGGGTS (SEQ ID NO. 26). The corresponding DNA sequence will be: 5′-CTC GAC CCA AAG AGC TGC GAG CGG AGC CAC AGC TGC CCA CCG TGC GGG GGT GGG TCC GGC GGT GGC ACT AGT-3′ (SEQ ID NO. 27). This sequence will be chemically synthesized and amplified by PCR using the following primers:

Primer 9 (forward): 5′-GTGG GCT AGC CTC GAC CCA AAG AGC TGC-3′ (SEQ ID NO. 28); the underlined sequence is the Nhe I site.

Primer 10 (reverse): 5′-AGG TTC TCG GGG CTG CCC ACT AGT GCC ACC GCC GGA CC-3′ (SEQ ID NO. 29).

Synthesis of the human C_{H}3 fragment. The vector containing the humanized SWLA3 monoclonal antibody sequence will be used as the template for generating the human C_{H}3 gene fragment by PCR. The following primers will be used in the PCR reaction.

Primer 11 (forward): 5′-GGG CAG CCC CGA GAA CAA C-3′ (SEQ ID NO. 30)

Primer 12 (reverse): 5′-GGT GGT CTG CAG TTT ACC CGG GGA CAG GGA GAG-3′ (SEQ ID NO. 31); the underlined sequence is a Pst I restriction site.

Assembling of the fragments to generate a peptide-minibody fusion protein gene. The DNA fragments encoding the protegrin, VL, VH, CH3, and the linkers will be assembled as diagramed below:

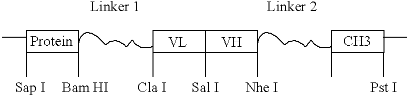

The fragment will be digested with Sap I and Pst I, and cloned into pTYB11 at the same restriction sites.

### Example 3

### Construction of Chimeric Construct Containing Surface-Binding Peptide And Anti-microbial Peptide

In addition to antibodies, some small peptide can also bind to surface structures of microorganisms or eukaryotic cells. These peptides, which we term “docking moiety”, allow more flexibility for the antimicrobial peptides (the killing moiety) to insert into the cell membrane for killing. These peptide are entirely man-made by combinatorial chemistry. Phage-display libraries of 8-12 amino acids peptide are commercially available. In this experiment, we have screened these libraries for peptides capable of specifically binding to a target organism, which can be bacteria, yeast, or other fungi. One or more of these peptides will then be fused to the anti-microbial peptide via a peptide linker, and expressed in an appropriate host.

Screening of phage display library for specific peptide binding to S. mutans, and C. albicans. A 12-amino acid peptide library (Ph.D-12) can be purchased from New England Biolabs. S. mutans will be grown anaerobically in TH medium at 37° C. overnight. Cells will be spun down and washed with PBS buffer. 10⁸ S. mutans cells will be mixed with 10¹⁰ CFU from the phage display library and incubated at room temperature for 10 min with gentle shaking. The mixture will be spun down in a microcentrifuge and the supernatant, which contains the unbound phage, will be transferred to a new tube and mixed with 10⁸ yeast cells for another round of binding (in this case, we recycle the phage particles that do not bind to S. mutans, and select for those that bind to yeast. The same process can go on for as many bacterial target as we desire). After binding, the bacterial or yeast cells will be washed 10 times with PBS and the bound phage will be eluted with 0.2 M glycine plus 1 mg/ml BSA (pH. 2.2). The eluent will be neutrolized with ⅙ vol of 1 M Tris.-HCl, pH 9.1, and amplified in an E. coli host strain for 4.5 h. The phage will be isolated by PEG precipitation, and used for the second round of binding as described for the first round. The entire process can be repeated 3 to 4 times to concentrate for phages carrying the peptides with the highest binding affinity for a bacterial or yeast cell. DNA sequence encoding these peptides can be obtained by sequencing the DNA contained in these specific phage particles. The DNA fragment will then be fused with the gene encoding the antimicrobial peptide by PCR manipulations, and cloned into an appropriate expression vector.

### Example 4

### Construction of an Expression System for Production of Minibody in Yeast

A yeast protein expression system is commercially available. In such system, an amino acid sequence encoding the protein of interest is fused to the pheromon factor α at the N-terminus and the myocin-his tag at the C-terminus. Such fusion protein is expressed, secreted outside of the cell and processed at the α factor cleavage site. The resulting protein is then purified by nickel column, which binds to the his-tag. The problem with this system is that the fusion protein of interest will have an added myocin-his tail at its C-terminus in the final product. If the protein is a minibody, this added tail could cause a problem in its mammalian application.

A bacterial system that allows fusion protein to be excised at the exact N- or C-terminus is also commercially available. This system uses an autocatalytic protein, intein, and a chitin-binding domain for purification. While this system may be ideal for producing the anti-microbial peptide alone, it lacks the proper modification required for minibody production.

We will combine the two systems to generate a new minibody-peptide fusion protein production system in yeast that will allow exact processing of the fusion protein and proper modification of the minibody moiety. Briefly, the DNA fragment encoding the intein-CBD fusion will be PCR amplified from the bacterial vector and cloned into the yeast vector downstream of the α-factor processing site. The DNA fragment encoding the minibody-peptide fusion will be fused with the intein domain and expressed as an intein-CBD-minibody fusion. This fusion complex will be secreted to the outside of the cell via the α-factor signal peptide, and purified from the culture supernatant by chitin affinity column. The minibody-peptide fusion protein will then be separated from the intein by automatic cleavage under reducing conditions.

Although the invention has been described with reference to the presently preferred embodiment, it should be understood that various modifications can be made without departing from the spirit of the invention. Accordingly, the invention is limited only by the following claims.

What is claimed is:

1. A composition useful for treatment of microbial organisms comprising
a targeting moiety and
an anti-microbial peptide moiety,
wherein the targeting moiety is coupled to the anti-microbial peptide moiety and recognizes a target microbial organism and wherein the composition has an anti-microbial effect on the target microbial organism.

2. The composition of claim 1, wherein the targeting moiety is a peptide.

3. The composition of claim 2, wherein the targeting moiety is coupled to the anti-microbial peptide moiety via a peptide linker.

4. The composition of claim 1, wherein the targeting moiety is a minibody.

5. The composition of claim 1, wherein the targeting moiety is selected from a group consisting of a scFv, minibody, Di-miniantibody, Tetra-miniantibody, (scFv)₂, Diabody, scDiabody, Triabody, Tetrabody, and Tandem diabody.

6. The composition of claim 1, wherein the targeting moiety comprises all or a portion of a variable region of an antibody.

7. The composition of claim 6, wherein the antibody is a monoclonal antibody specific to S. mutans.

8. The composition of claim 7, wherein the antibody is selected from the group consisting of SWLA1, SWLA2, and SWLA3.

9. The composition of claim 1, wherein the targeting moiety comprises a variable region of a light chain and a variable region of a heavy chain of an antibody.

10. The composition of claim 9, wherein the targeting moiety further comprises a constant domain.

11. The composition of claim 10, wherein the constant domain is connected to the variable region of the heavy chain by a peptide linker.

12. The composition of claim 10 comprises a dimer, wherein each monomer of the dimer comprises a fusion polypeptide containing the targeting moiety and the anti-microbial peptide moiety.

13. The composition of claim 1, wherein the targeting moiety is a ligand to a receptor of the target microbial organism.

14. The composition of claim 1, wherein the anti-microbial peptide moiety comprises a peptide selected from the group consisting of alexomycin, andropin, apidaecin, bacteriocin, β-pleated sheet bacteriocin, bactenecin, buforin, cathelicidin, α-helical clavanin, cecropin, dodecapeptide, defensin, β-defensin, α-defensin, gaegurin, histatin, indolicidin, magainin, nisin, protegrin, ranalexin, and tachyplesin.

15. The composition of claim 1, wherein the anti-microbial peptide moiety comprises histatin 5.

16. The composition of claim 1, wherein the anti-microbial peptide moiety comprises a peptide comprising an amino acid sequence as shown in SEQ ID NO. 2.

17. The composition of claim 1, wherein the anti-microbial peptide moiety comprises dhvar 1.

18. The composition of claim 1, wherein the anti-microbial peptide moiety comprises a peptide comprising an amino acid sequence as shown in SEQ ID NO. 6.

19. The composition of claim 1, wherein the anti-microbial peptide moiety comprises protegrin PG-1.

20. The composition of claim 1, wherein the anti-microbial peptide moiety comprises a peptide comprising an amino acid sequence as shown in SEQ ID NO. 15.

21. The composition of claim 1, wherein the anti-microbial peptide moiety comprises Novispirin G10.

22. The composition of claim 1, wherein the anti-microbial peptide moiety comprises a peptide comprising an amino acid sequence as shown in SEQ ID NO. 17.

23. The composition of claim 1, wherein the target microbial organism is selected from the group consisting of bacteria, ricketsia, fungi, yeasts, protozoa, and parasites.

24. The composition of claim 1, wherein the target microbial organism is a cariogenic organism.

25. The composition of claim 1, wherein the target microbial organism is Streptococcus mutans.

26. The composition of claim 25, wherein the anti-microbial peptide moiety comprises a peptide selected from the group consisting of histatin 5, dhvar 1, protegrin PG-1, and Novispirin G10.

27. The composition of claim 1, wherein the target microbial organism is selected from the group consisting of Escherichia coli, Shigella dysenteriae, Salmonella typhimurium, Streptococcus pneumoniae, Staphylococcus aureus, and Pseudomonas aeruginosa.

28. The composition of claim 27, wherein the anti-microbial peptide moiety comprises a peptide selected from the group consisting of buforin, cecropin, indolicidin, and nisin.

29. The composition of claim 1, wherein the target microbial organism is selected from the group consisting of Escherichia coli, Shigella dysenteriae, Salmonella typhimurium, Streptococcus pneumoniae, Staphylococcus aureus, Pseudomonas aeruginosa, Candida albicans, Cryptococcus neoformans, Candida krusei, and Helicobacter pylori.

30. The composition of claim 29, wherein the anti-microbial peptide moiety comprises a peptide selected from the group consisting of magainin and renalexin.

31. The composition of claim 1, wherein the target microbial organism is herpes simplex virus and the anti-microbial peptide moiety comprises a peptide of magainin.

32. The composition of claim 1, wherein the target microbial organism is selected from the group consisting of Streptococcus mutans, Neisseria gonorrhoeae, Chlamydia trachomatis,and Haemophilius ducreyi and wherein the anti-microbial peptide moiety comprises a peptide of protegrin.

33. The composition of claim 1, wherein the target microbial organism is selected from the group consisting of Camphylobacter jejuni, Moraxella catarrhalis, and Haemophilius influenzae and wherein the anti-microbial peptide moiety comprises a peptide of alexomycin.

34. The composition of claim 1, wherein the target microbial organism is
Streptococcus pneumoniae and the anti-microbial peptide moiety is selected from the group consisting of defensin, α defensin and β pleated sheet defensin.

35. A method of treating a target microbial organism infection comprising administering to a subject in need of such treatment an effective amount of the composition of claim 1.

36. The method of claim 35, wherein the target microbial organism infection is on a mucosal surface.

37. The method of claim 36, wherein the mucosal surface is selected from the group consisting of mouth, vagina, gastrointestinal tract, and esophageal tract.

38. The method of claim 35, wherein the target microbial organism infection is a S. mutans infection in a mouth.

39. The method of claim 38 comprising administering to a subject in need of such treatment an effective amount of the composition of claim 5.

40. The method of claim 38 comprising administering to a subject in need of such treatment an effective amount of the composition of claim 6.

41. The method of claim 38 comprising administering to a subject in need of such treatment an effective amount of the composition of claim 8.

42. The method of claim 38 comprising administering to a subject in need of such treatment an effective amount of the composition of claim 12.

43. The method of claim 37, wherein the target microbial organism infection is a Candida albicans infection in vagina.

44. The method of claim 37, wherein the target microbial organism infection is an infection in gastrointestinal tract selected from the group consisting of a Helicobacter pylori infection, Campylobacter jerjuni infection, Vibrio cholerae infection, salmonella infection, Shigella infection, and Escherichia coli infection.

45. The method of claim 37, wherein the target microbial organism infection is an oral infection selected from the group consisting of porphyromonas gingivalis, Actinomyces, Veillonella spirochetes, and gram-negative flora infection

46. The method of claim 37, wherein the target microbial organism infection is an Clostridium difficile infection in gastrointestinal tract or esophageal tract.

47. A method of making the composition of claim 1 comprising using an expression construct containing a sequence encoding the targeting moiety, the anti-microbial peptide moiety, pheromon factor α, intein, and chitin binding domain.

48. A method of making the composition of claim 2 comprising using an expression construct containing a sequence encoding the targeting moiety, the anti-microbial peptide moiety, pheromon factor α, intein, and chitin binding domain.
